(19) United States
(12) Patent Application Publication
(10) Pub. No.: US 2024/0316247 A1
(43) Pub. Date: Sep. 26, 2024

(54) Medical Graft Devices And Methods Of Making Thereof

(71) Applicant: ACell, Inc., Columbia, MD (US)

(72) Inventors: Andrea S. Marks, Takoma Park, MD (US); Nathaniel Remlinger, Baltimore, MD (US); Alexander M. LeBrun, Sykesville, MD (US)

(21) Appl. No.: 18/259,461

(22) PCT Filed: Dec. 17, 2021
(86) PCT No.: PCT/US2021/064119

§ 371 Date: Jun. 27, 2023

Related U.S. Application Data
(60) Provisional application No. 63/131,559, filed on Dec. 29, 2020.

(51) Int. Cl. A61L 27/36; A61K 35/38; A61L 27/56

(52) U.S. Cl. CPC A61L 27/3633; A61K 35/38; A61L 27/56

(57) ABSTRACT

Large-area medical graft devices comprise at least two sheets of extracellular matrix material (ECM). The sheets of ECM have overlapping regions that are compressed together during a non-drying process, and other overlapping regions that are not compressed together. The compressed overlapping regions have a more collapsed matrix structure, while the non-compressed overlapping regions having a more open matrix structure. The sheets of ECM can have a stacked orientation to create a desired thickness, or a staggered orientation to create a desired surface area, or combinations of both orientations.

### CROSS-REFERENCE TO RELATED APPLICATIONS

This application claims priority to and benefit under 35 U.S.C. 119(e) to U.S. Provisional Application No. 63/131,559, filed Dec. 29, 2020, the entire contents of which are incorporated by reference herein.

### TECHNICAL FIELD

The disclosure relates to medical graft devices comprising an extracellular matrix having regions of differing properties.

### BACKGROUND

Medical graft devices made from dried sheets of bioabsorbable tissue comprising an extracellular matrix (ECM) can serve as tissue grafts and in wound healing applications. Common methods of preparing ECM-based collagenous tissues for use as medical graft devices include vacuum pressing and lyophilization (freeze drying). Both methods produce sheets of ECM tissue having differing properties, such as degree of porosity or of compression of the matrix structure.

For example, vacuum pressing comprises compressing hydrated, remoldable material while subjecting the material to a vacuum. Tissue compressed by vacuum pressing generally has a higher tensile strength and lower strain value compared to tissue compressed by other methods because of its more compressed matrix structure. Vacuum pressing can also laminate multiple layers of ECM material together by crushing the matrix structure of the ECM. Lyophilization, on the other hand, comprises drying tissue by sublimation, a process of changing ice crystals from a solid directly to a gas without passing through an intermediate liquid phase. During lyophilization, a vacuum applied to frozen tissue at low temperatures causes the ice crystals to sublimate from the frozen tissue, leaving behind small pockets of open space formerly occupied by the ice. Hence, the resultant dried tissue has a more open matrix structure compared to tissue dried by other methods due to pores created in the frozen tissue after extraction of the ice crystals. Surgeons often use lyophilized devices for wound management since their open matrix structure results in a faster resorption time and their open spaces allow for increased integration and interaction with local cells and tissue.

Problematically, with the traditional lyophilization process, the size and thickness of the underlying tissue source (for example, intestinal tissue) typically limits the size and thickness of sheets of ECM available for use as medical graft device. The lyophilization process alone does not create lamination between tissue layers that allow for the creation of devices larger than the area of a single piece of tissue alone. Hence, a typical sheet produced from the UBM ECM source may be no larger than 10×15 cm after processing as a single UBM sheet does not typically exceed the 10×15 cm area, meaning that a surgeon may need to use multiple sheets to apply to a large wound area. Furthermore, the surgeon may need to apply several layers of the sheets to achieve a desired thickness. Therefore, a need exists for larger area and/or thicker lyophilized medical graft devices. However, as introduced above, lyophilization alone cannot produce such ECM devices, since lyophilization does not laminate layers of tissue together with a high enough lamination strength for viable devices. Thus, a continuing need exists for large-area medical graft devices having the open-matrix structure of a lyophilized device and method of creation.

### SUMMARY

The present disclosure describes large-area medical graft devices comprising at least two sheets of planar biological material, such as extracellular matrix material (ECM). The sheets of ECM have overlapping regions that are compressed together during a non-drying process, and other overlapping regions that are not compressed together. Once the appropriate overlapping regions of the sheets are compressed together, the entire device is lyophilized to bond the compressed regions together, resulting in the compressed overlapping regions having a more collapsed matrix structure and the non-compressed overlapping regions having a more open matrix structure. Advantageously, the sheets can have a stacked orientation to create a desired thickness, or a staggered orientation to create a desired surface area. The sheets can also have combinations of both a stacked and a staggered orientation to create both a desired surface area and thickness.

In examples, medical graft devices of the disclosure include at least two sheets of extracellular matrix material (ECM) having at least one first overlapping region that is compressed together during a non-drying method, and at least one second overlapping region that is not compressed together. The at least one second overlapping region has a more open matrix structure than the at least one first overlapping region. The compressed at least one first overlapping region is bonded together during a drying method.

In further examples, the at least two sheets of ECM are at least three sheets of ECM. In examples, the at least two sheets of ECM are configured in a stacked orientation or in a staggered orientation. In examples, the staggered orientation comprises at least a first layer of the at least two sheets of ECM, and at least a second layer of the at least two sheets of ECM is offset from the first layer. In examples, the ECM is synthetic or naturally-occurring. In examples, the at least one first overlapping region extends around a periphery of the device and at least partially defines the at least one second overlapping region. In examples, the at least one first overlapping region has a cross-bar configuration and at least partially defines the at least one second overlapping region. In examples, the medical graft device is a tissue graft. In other examples, the medical graft device is a wound dressing device.

Examples of a method of making a medical graft device of this disclosure include preparing at least two hydrated sheets of extracellular matrix material (ECM). Regions of the at least two hydrated sheets are then compressed without drying. The at least two hydrated sheets are then frozen to bond together the compressed regions of the at least two hydrated sheets. Non-compressed regions of the at least two dehydrated sheets have a more open matrix structure than the compressed regions of the at least two dehydrated sheets.

In further examples, preparing the at least two hydrated sheets includes securing the at least two hydrated sheets to a first support structure having at least one open area defined by a semi-solid area, and placing a second support structure over the at least two hydrated sheets. The second support structure has an identical geometry to the first support structure. The second support structure is placed over the at least two hydrated sheets such that at least one open area and a semi-solid area of the second support structure aligns with the at least one open area and the semi-solid area of the first support structure. In examples, compressing the regions of the at least two hydrated sheets includes compressing regions of the at least two hydrated sheets in contact with the semi-solid areas of the first and second support structures. In examples, the at least two hydrated sheets are secured to the first support structure in a stacked orientation or in a staggered orientation. In examples, securing the at least two hydrated sheets to the first support structure includes securing the at least two hydrated sheets to securing elements disposed about an outer edge of the first support structure. In examples, after dehydrating the at least two hydrated sheets, the second support structure is removed from the at least two sheets. In examples, after dehydrating the at least two hydrated sheets, the at least two dehydrated sheets are removed from the first support structure. In examples, securing the at least two hydrated sheets to the first support structure includes securing at least a first one of the at least two hydrated sheets to the first support structure in a first layer, securing at least a second one of the at least two hydrated sheets to the first support structure in a second layer offset from the first layer, and securing at least a third one of the at least two hydrated sheets to the first support structure in a third layer aligned with the first layer.

In examples, the medical graft devices of this disclosure also include medical graft devices produced by preparing at least two hydrated sheets of extracellular matrix material (ECM), compressing regions of the at least two hydrated sheets without drying, and freezing the at least two hydrated sheets to bond together the compressed regions of the at least two hydrated sheets. Non-compressed regions of the at least two dehydrated sheets have a more open matrix structure than the compressed regions of the at least two dehydrated sheets.

A reading of the following detailed description and a review of the associated drawings will make apparent the advantages of these and other features. Both the foregoing general description and the following detailed description serve to explain the disclosure only and do not restrict aspects of the disclosure as claimed.

### BRIEF DESCRIPTION OF THE DRAWINGS

Reference to the detailed description, in conjunction with the following figures, will make the disclosure more fully understood, wherein:

FIG. 1 illustrates an example of the medical graft device of this disclosure;

FIGS. 2A and 2B illustrate a method of assembling sheets of ECM material to create the medical graft devices of this disclosure in a stacked orientation (FIG. 2A) and a staggered orientation (FIG. 2B);

FIG. 3 is a flow chart of the steps of making the medical graft devices of this disclosure;

FIGS. 4A-G illustrate the preparation step of the method of making the medical graft devices of this disclosure;

FIGS. 5A-C illustrate the compression step of the method of making the medical graft devices of this disclosure;

FIG. 6 illustrates the preliminary freezing step of the method of making the medical graft devices of this disclosure;

FIG. 7 illustrates the lyophilization step of the method of making the medical graft devices of this disclosure;

FIG. 8A is a top plan view of an example of the medical graft device of this disclosure; and

FIG. 8B is a cross-sectional view taken at section line A-A of FIG. 8A.

### DETAILED DESCRIPTION

In the description that follows, like components have the same reference numerals, regardless of whether they are present in different examples. To illustrate examples in a clear and concise manner, the drawings may not necessarily illustrate scale and may show certain features in somewhat schematic form. Features described and/or illustrated with respect to one example may exist in the same way or in a similar way in one or more other examples and/or in combination with or instead of the features of the other examples.

As used in the specification and claims, for the purposes of describing and defining the invention, the terms “about” and “substantially” represent the inherent degree of uncertainty attributed to any quantitative comparison, value, measurement, or other representation. The terms “about” and “substantially” also represent the degree by which a quantitative representation may vary from a stated reference without resulting in a change in the basic function of the subject matter at issue. “Comprise,” “include,” “have,” and variations of each word include the listed parts and can include additional parts not listed. “And/or” includes one or more of the listed parts and combinations of the listed parts. The terms “upper,” “lower,” “above,” “below,” and the like serve to clearly describe the disclosure but do not limit the structure, positioning and/or operation of the disclosure in any manner. As used in the specification and claims, the term “laminate” describes both the process and the end result of two or more independent pieces of tissue bonding together. Thus, lamination produces a directed area of connection between the tissues that would not occur unless intentionally created.

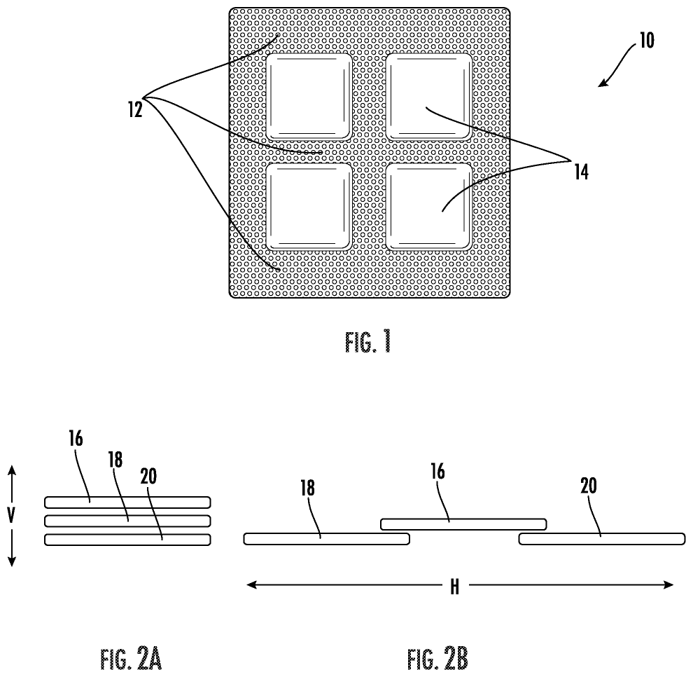

FIG. 1 shows an example of a medical graft device 10 of this disclosure made from sheets of a bioresorbable, extracellular matrix (ECM). Examples of the ECM material may be synthetic ECM (for example, synthetic collagen, or engineered collagen described in U.S. Pat. No. 6,969,523, which is incorporated herein by reference) or naturally-occurring ECM (for example, decellularized animal tissue such as submucosa of the intestine (SIS), dermis, urinary bladder, or liver). The ECM may be derived from native mammalian tissues including but not limited to submucosa, dermis, epithelial basement membrane, aponeurosis, fascia, tendon, ligament, smooth and skeletal muscle and treatment site-specific ECM. The native mammalian tissue source may be porcine, bovine, ovine, allogenic, or autogenic. Some non-limiting examples of naturally-occurring ECM may include the urinary bladder matrix (UBM) described in U.S. Pat. No. 6,576,265, which is incorporated herein by reference, and acellular dermal matrices described in U.S. Pat. No. 9,011,895, which is incorporated herein by reference. Non-limiting examples of preparing solid sheets and strips of ECM material may include the methods described in U.S. Pat. No. 5,711,969, incorporated herein by reference. In an exemplary embodiment, the ECM used in deice 10 is a urinary bladder matrix which has been processed to remove cellular content and comprises epithelial basement membrane and lamina propria.

The device 10 had laminated regions 12 of compressed tissue with a collapsed matrix structure, and non-laminated regions 14 of uncompressed tissue that preserves the native matrix structure. The device 10 thus advantageously combines the laminated properties of multi-layer tissue produced by vacuum pressing with the open matrix structure of lyophilized tissue.

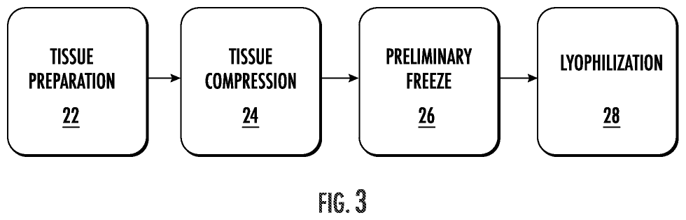

As shown in FIGS. 2A and 2B, the device 10 comprises at least two sheets 16, 18, 20 of hydrated ECM tissue layered in either a stacked orientation (FIG. 2A) or a staggered orientation (FIG. 2B). The stacked orientation comprises three hydrated sheets 16, 18, 20 placed on top of one another in a vertical direction V, while the staggered orientation comprises hydrated sheets 16, 18, 20 placed with overlapping regions primarily in a horizontal direction H. Stacking creates devices 10 with multiple layers, while staggering creates devices 10 with larger areas, as further described below.

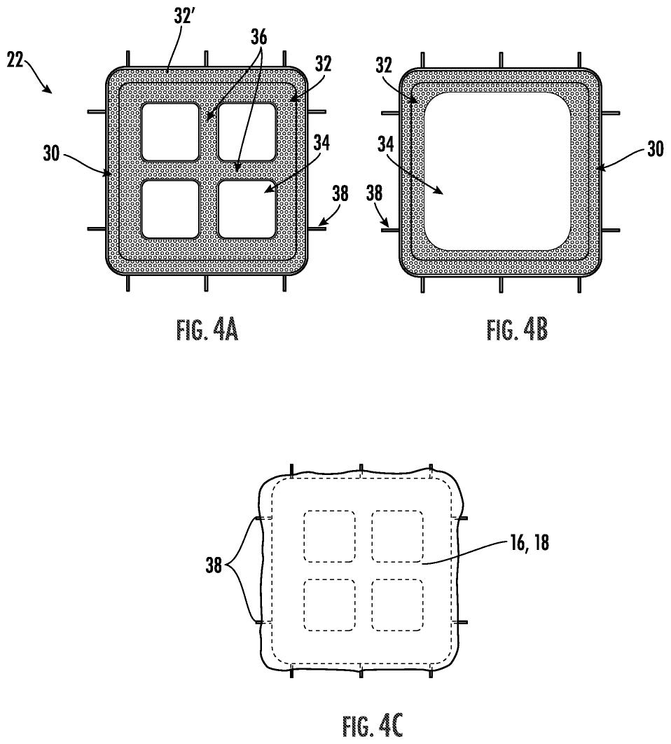

FIG. 3 illustrates exemplary steps for making the devices 10 of this disclosure. The steps include tissue preparation 22, tissue compression without drying 24, preliminary freeze 26, and lyophilization 28. The disclosure describes each of these steps in more detail below.

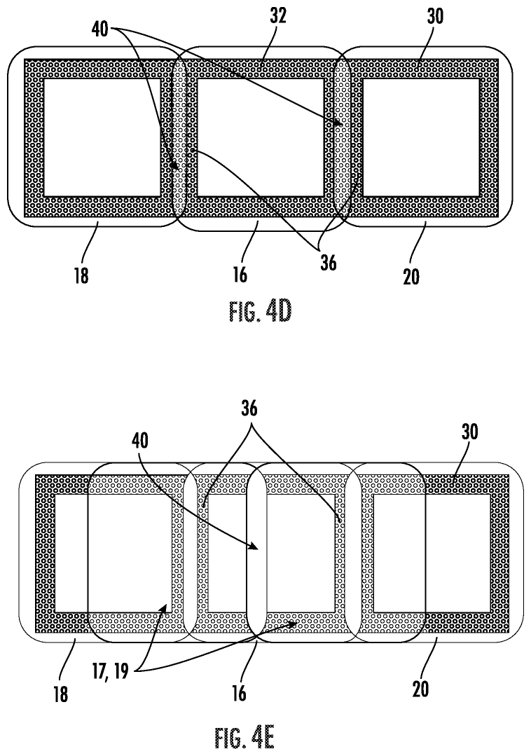

Turning now to FIGS. 4A and 4B, in the initial step of tissue preparation 22, wet sheets of decellularized and disinfected ECM are wrapped onto a first porous board 30. A porous board may also be referred to as a perforated board, a fluid permeable board, or a permeable board or structure. In examples, the first porous board 30 comprises a material subjectable to additive manufacturing, such as a thermoplastic. However, any suitable material that contains pores configured to allow transfer of water and heat from one side of the board to the other, including for example fluid flow during a lyophilization process, can form the first porous board 30. The first porous board 30 can have any suitable shape—for example, square, rectangular, round or polygonal. The first porous board 30 comprises a perimeter area 32 defining at least one open area 34. In examples, the perimeter area is semi-solid (i.e., not sealed) and may optionally include one or more raised or recessed areas which differ in thickness from the rest of the porous board, for example, by about 1 mm, to increase or decrease regional compression as desired. In the example shown in FIG. 4A, an outer section 32′ of the perimeter area 32 is raised slightly than the rest of perimeter area 32 to increase compression near the rim of the final device 10. In examples, the first porous board 30 comprises a plurality of open areas 34 (FIG. 4A) having semi-solid crossbars 36 between the open areas 34, or only one open area 34 (FIG. 4B). The first porous board 30 may also include a plurality of securing elements 38, such as nails, placed evenly around an outer edge of the perimeter area 32. In examples, the first porous board 30 may include additional crossbars 36 or additional open areas 34 depending on the geometry and size of laminated regions desired in the final device 10. As long as there are crossbars over each ECM overlap, the device can theoretically be as large as desired, including for example, larger than 10×15 cm.

As shown in FIG. 4C, in the stacked orientation, at least two hydrated sheets 16, 18 of ECM material are wrapped onto a top surface of the first porous board 30 such that the securing elements 38 pierce the outer edges of the sheets 16, 18. The securing elements 38 keep the sheets 16, 18 taught and in place as additional sheets are optionally layered on top of the sheets 16, 18. As shown in FIG. 4D, in the staggered orientation, a least two hydrated sheets 16, 18, 20 of ECM material are layered onto a top surface of the first porous board 30 such that the overlapping regions 40 of the sheets 16, 18, 20 align with the crossbars 36 of the first porous board 30. This alignment ensures that the overlapping regions 40 will laminate together in the final device. The securing elements 38 (not shown) pierce the outer edges of the sheets 16, 18, 20 around the perimeter area 32 to hold the sheets 16, 18, 20 in place. Optionally, as shown in FIG. 4E, a second layer of hydrated sheets 17, 19 may be layered on top of sheets 16, 18, 20 such that the overlapping regions 40 of the sheets 17, 19 are offset with respect to the crossbars 36 of the first porous board 30. Notably, if an offset layer is used, a final layer of hydrated sheets (not shown) must be placed over the offset layer such that the overlapping regions of the third layer of sheets align with the crossbars 36 of the first porous board 30 to prevent delamination of the final device. As shown in FIGS. 4F and 4G, once the desired number of layers of hydrated sheets 16, 18 are wrapped onto the first porous board 30, a second porous board 42, having an identical geometry to the first porous board 30 but without securing elements 38, is placed on top of the hydrated sheets 16, 18 such that both the open areas 34 and the solid perimeter areas 32 of the first and second porous boards 30, 42 align. FIG. 4G is a side view of the assembly shown in FIG. 4F from a corner thereof. The porous board 30 may optionally have raised or recessed regions, such as recesses 52 that correspond to any clamps or other means used for compression, but such raised or recessed regions are optional and not essential for the process.

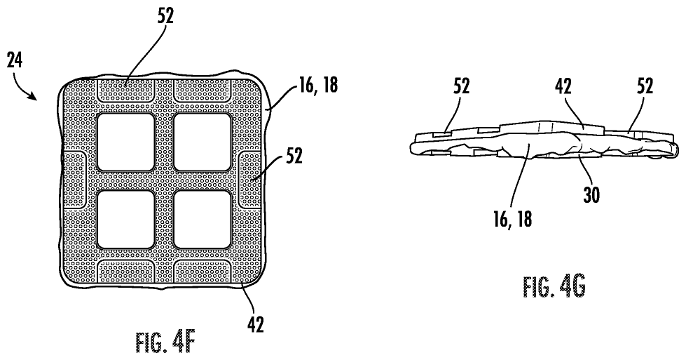

FIGS. 5A-C illustrate the subsequent tissue compression step 24. A mechanical force in the form of a vacuum or other sealed bag 44 (FIGS. 5A and 5B), clamps 46 (FIG. 5C), or other suitable means applies consistent pressure across the surfaces of the porous boards 30, 42 without drying the hydrated sheets 16, 18 to create a compressed tissue/porous board assembly 48. Use of the sealed bag 44, in particular, creates a closed system preventing leakage of water from the hydrated sheets 16, 18. During the compression step 24, only the regions of the hydrated sheets 16, 18 in direct contact with or positioned between the crossbars 36 or the perimeter areas 32 of the porous boards 30, 42 will compress. Regions of the hydrated sheets 16, 18 aligning with the open areas 34 of the porous board 30, 42 will not compress. The compression step 24 crushes the ECM structure of the hydrated sheets 16, 18 within the selected regions to initiate physical van der Waals forces within the tissue layers. The greater the compression force, the greater the degree of the van der Waals forces between the hydrated sheets 16, 18, yielding a higher degree of lamination in the final device 10.

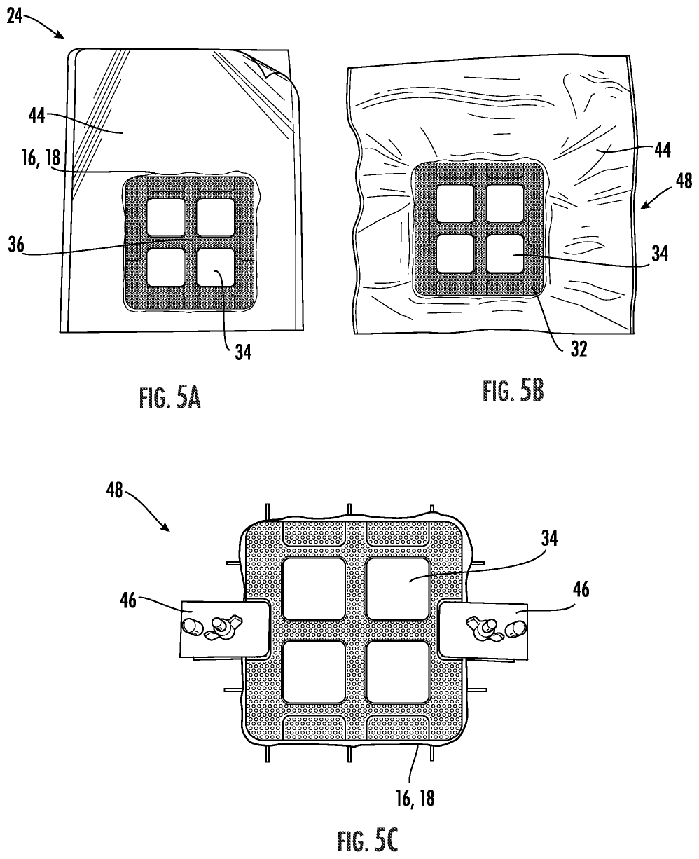

FIG. 6 illustrates the subsequent preliminary freezing step 26. The compressed tissue/porous board assembly 48 is placed in a freezer immediately after the tissue compression step 24 to freeze the hydrated sheets 16, 18 in a compressed state before drying. The sheets 16, 18 may be frozen by placing the entire assembly 48 into a freezer, dipping the assembly 48 into a bath of liquid nitrogen, or other suitable means of freezing. FIG. 6 illustrates an example of the frozen assembly 48 using the compression technique of the sealed bag 44, and the freezing technique of a below-zero freezer (not shown). The duration of the preliminary freezing step 26 may vary depending on the desired size of the ice crystals and resultant pores in the final device 10.

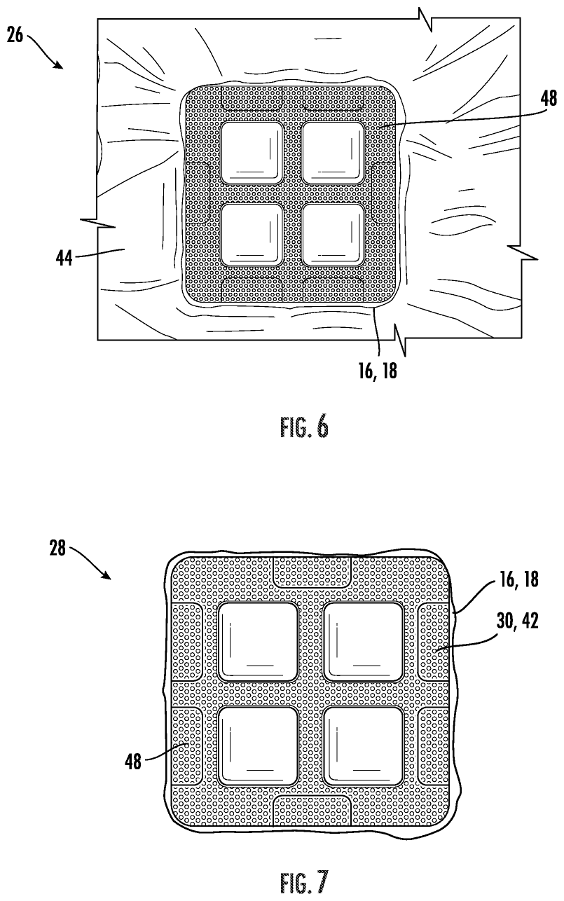

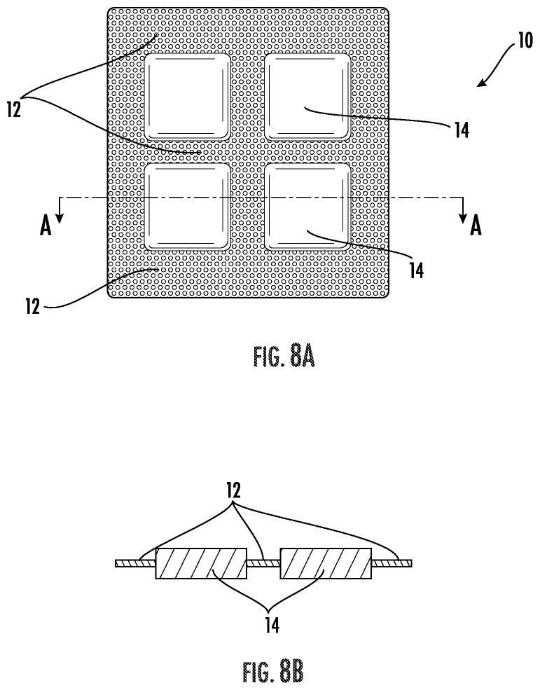

FIG. 7 illustrates the final lyophilization step 26. Before placing the tissue/porous board assembly 48 in a lyophilizer, any accessory items used to provide compression on the assembly 48 are removed, including the sealed bag 44 or clamps 46, leaving only the porous boards 30, 42. The porous boards 30, 42 do not provide any particular purpose within the lyophilizer but merely are adhered to the frozen sheets 16, 18 by ice crystals prior to placement in the lyophilizer. Before placing the assembly 48 in the lyophilizer, the lyophilizer is pre-chilled to minimally −20° C. The assembly 48 is then lyophilized (for example, overnight) to dry the sheets 16, 18 and to produce the final medical graft device 10 (FIG. 8A).

In the methods of the present disclosure, which may be referred to as lyophilization compression methods, the compressed matrix structure is a result of the compression of the porous boards during the tissue compression step 22. This compression can be achieved through the use of the vacuum sealing in a bag or clamps as illustrated in the drawings and described above, or other means of pressing the boards together. As illustrated in FIG. 8B, laminated regions 12 comprise a compressed matrix structure generated by means of the compressive forces of the porous boards. Non-laminated regions 14 are regions where the tissue was not in direct contact with the porous board. Non-laminated regions 14 have a preserved matrix structure, which is a more open matrix structure than the matrix structure of laminated regions 12. Laminated regions 12 have a more collapsed matrix structure than the matrix structure of the non-laminated regions 14. Laminated regions 12 also have a more collapsed matrix structure than the native matrix structure of the tissue before it is processed. The tissue matrix structure is generally maintained or preserved after the tissue is lyophilized. The process of lyophilization maintains the native matrix structure. In other words, the original porosity and thickness of the tissue is preserved in areas that are uncompressed. Thus, the porosity of the UBM is a function of the tissue matrix structure. The matrix structure is either maintained or manipulated by compression during processing. The preservation or compression of the tissue matrix can be assessed by scanned electron microscope (SEM) imaging.

The medical graft device 10 can be used, for example, as a tissue graft or wound dressing device, or to create a pouch structure for encasing an implantable electronic device.

One skilled in the art will realize the disclosure may embody other specific forms without departing from the spirit or essential characteristics thereof. The foregoing examples in all respects illustrate rather than limit the disclosure described herein. The appended claims, rather than the foregoing description, thus indicate the scope of the disclosure, and embrace all changes that come within the meaning and range of equivalency of the claims.

1. A medical graft device comprising:
at least two sheets of extracellular matrix material (ECM) having at least one first overlapping region that is compressed together during a non-drying method, and at least one second overlapping region that is not compressed together, the at least one second overlapping region having a more open matrix structure than the at least one first overlapping region;
wherein the compressed at least one first overlapping region is bonded together during a drying method.

2. The medical graft device of claim 1, wherein the at least two sheets of ECM are at least three sheets of ECM.

3. The medical graft device of claim 1, wherein the at least two sheets of ECM are configured in a stacked orientation.

4. The medical graft device of claim 1, wherein the at least two sheets of ECM are configured in a staggered orientation.

5. The medical graft device of claim 4, wherein the staggered orientation comprises at least a first layer of the at least two sheets of ECM, and at least a second layer of the at least two sheets of ECM offset from the first layer.

6. The medical graft device of claim 1, wherein the ECM is synthetic or naturally-occurring.

7. The medical graft device of claim 1, wherein the at least one first overlapping region extends around a periphery of the device and at least partially defines the at least one second overlapping region.

8. The medical graft device of claim 1, wherein the at least one first overlapping region has a cross-bar configuration and at least partially defines the at least one second overlapping region.

9. The medical graft device of claim 1, wherein the medical graft device is a tissue graft.

10. The medical graft device of claim 1, wherein the medical graft device is a wound dressing device.

11. A method of making a medical graft device, comprising:
preparing at least two hydrated sheets of extracellular matrix material (ECM);
compressing, without drying, regions of the at least two hydrated sheets;
freezing the at least two hydrated sheets; and
dehydrating the at least two hydrated sheets to bond together the compressed regions of the at least two hydrated sheets;
wherein non-compressed regions of the at least two dehydrated sheets have a more open matrix structure than the compressed regions of the at least two dehydrated sheets.

12. The method of claim 11, wherein preparing the at least two hydrated sheets comprises:
securing the at least two hydrated sheets to a first support structure having at least one open area defined by a semi-solid area; and
placing a second support structure over the at least two hydrated sheets;
wherein the second support structure has an identical geometry to the first support structure; and
wherein the second support structure is placed over the at least two hydrated sheets such that at least one open area and a semi-solid area of the second support structure aligns with the at least one open area and the semi-solid area of the first support structure.

13. The method of claim 12, wherein compressing the regions of the at least two hydrated sheets comprises compressing regions of the at least two hydrated sheets in contact with the semi-solid areas of the first and second support structures.

14. The method of claim 12, wherein the at least two hydrated sheets are secured to the first support structure in a stacked orientation.

15. The method of claim 12, wherein the at least two hydrated sheets are secured to the first support structure in a staggered orientation.

16. The method of claim 12, wherein securing the at least two hydrated sheets to the first support structure comprises securing the at least two hydrated sheets to securing elements disposed about an outer edge of the first support structure.

17. The method of claim 12, further comprising, after dehydrating the at least two hydrated sheets, removing the second support structure from the at least two sheets.

18. The method of claim 12, further comprising, after dehydrating the at least two hydrated sheets, removing the at least two dehydrated sheets from the first support structure.

19. The method of claim 12, wherein securing the at least two hydrated sheets to the first support structure comprises:
securing at least a first one of the at least two hydrated sheets to the first support structure in a first layer;
securing at least a second one of the at least two hydrated sheets to the first support structure in a second layer offset from the first layer; and
securing at least a third one of the at least two hydrated sheets to the first support structure in a third layer aligned with the first layer.

20. A medical graft device produced by the method of claim 11.
